(19) United States
(12) Patent Application Publication
(10) Pub. No.: US 2023/0210064 A1
(43) Pub. Date: Jul. 6, 2023

(54) ORGANIC PLANTING METHOD OF GASTRODIA ELATA

(71) Applicant: Southwest Forestry University, Kunming City (CN)

(72) Inventors: Xiahong HE, Kunming City (CN); Rui SHI, Kunming City (CN); Shu HE, Kunming City (CN); Bingjie XIONG, Kunming City (CN); Youguo HUANG, Kunming City (CN); Xingru YAN, Kunming City (CN); Ao ZHANG, Kunming City (CN); Xixi LIANG, Kunming City (CN)

(73) Assignee: Southwest Forestry University, Kunming City (CN)

(21) Appl. No.: 17/903,199

(22) Filed: Sep. 6, 2022

(30) Foreign Application Priority Data
Dec. 31, 2021 (CN) 202111660318.9

(51) Int. Cl. A01G 22/25; A01G 18/40; C12N 1/14

(52) U.S. Cl. CPC A01G 22/25; A01G 18/40; C12N 1/14

(57) ABSTRACT

The present disclosure relates to the technical field of Gastrodia elata planting, in particular to an organic planting method of G. elata, including the process of mycelia colonized material preparation: cutting a branch of Quercus acutissima into bars; cutting out fish scale openings on the bars to obtain bars with the fish scale openings; successively soaking the bars with the fish scale openings in boiling water and a growth-promoting solution to obtain bars after growth promotion treatment; and putting Amillariella mellea spawns and an inoculation substrate into each fish scale opening of the bars after the growth promotion treatment to obtain a mycelia colonized material. The growth-promoting solution and the inoculation substrate are prepared by treatment of fresh G. elata stalks with Bacillus pumilus and hot-dip extraction treatment in ethanol.

### CROSS-REFERENCE TO RELATED APPLICATION

This patent application claims the benefit and priority of Chinese Patent Application No. 202111660318.9, filed with the China National Intellectual Property Administration on Dec. 31, 2021, the disclosure of which is incorporated by reference herein in its entirety as part of the present application.

### TECHNICAL FIELD

The present disclosure relates to the technical field of Gastrodia elata planting, in particular to an organic planting method of G. elata.

### BACKGROUND

G. elata B1. is a perennial herb of the family Orchidaceae. Gastrodiae Rhizoma, one of the traditional Chinese herbal medicines in China, was first recorded in the name of Chijian in the Shennong's Classic of Materia Medica and rated as the top grade. The tuber of G. elata is usually used as medicine with very high medical and healthcare value. Gastrodiae Rhizoma contains gastrodin (GAS), p-hydroxybenzyl alcohol (HBA), polysaccharides, proteins, amino acids, and trace elements, among which GAS and HBA are primary active pharmaceutical ingredients (APIs) of the Gastrodiae Rhizoma and key indicators for evaluating the quality thereof. In the past, G. elata was mostly wild, but in recent years, artificial cultivation thereof has been successful. Because G. elata is a rootless plant, its tuber is buried underground throughout its growth cycle and relies on mycelia of Amillariella mellea to provide it with nutrients needed for growth, and moreover, the mycelia can only survive in cortical cells of its tuber but cannot penetrate further into the tuber of G. elata. After a growing rhizomorph of A. mellea touches G. elata, the growth point at the apex of the branch of the rhizomorph invades the protocorm or tuber of G. elata, and the symbiotic union of G. elata and A. mellea begins. The first step in the cultivation of G. elata is to cultivate a good mycelia colonized material for A. mellea, which requires rapid spawn running, vigorous growth and no contamination by weed molds. The mycelia colonized material for A. mellea has a substantial influence on the quality of artificially cultivated of G. elata, especially on the content of the functional ingredients of G. elata. Therefore, how to cultivate suitable mycelia colonized materials for A. mellea is the key to improving the quality of artificially cultivated G. elata.

### SUMMARY

An objective of the present disclosure is to provide an organic planting method of G. elata, so as to solve the technical problem of limited growth-promoting effect of a mycelia colonized material for A. mellea on G. elata in the prior art.

To achieve the above objective, the present disclosure adopts the following technical solution:

An organic planting method of G. elata is provided, including the process of mycelia colonized material preparation: cutting a branch of Quercus acutissima into bars; cutting out fish scale openings on the bars to obtain bars with the fish scale openings; successively soaking the bars with the fish scale openings in boiling water and a growth-promoting solution to obtain bars after growth promotion treatment; putting A. mellea spawns and an inoculation substrate into each fish scale opening of the bars after the growth promotion treatment to obtain a mycelia colonized material;

where the growth-promoting solution and the inoculation substrate are prepared by the following methods: cutting up a fresh G. elata stalk to obtain materials to be treated, spraying a Bacillus pumilus suspension on the materials to be treated, and conducting fermentation culture to obtain fermented materials; using ethanol as a solvent to conduct hot-dip extraction on the fermented material, and conducting solid-liquid separation to obtain residues and an extract; conducting moist heat sterilization, drying and pulverization on the residues to obtain the inoculation substrate; and conducting concentration, reconstitution and moist heat sterilization on the extract to obtain the growth-promoting solution.

The principle and advantages of this solution are as follows:

Fermentation of G. elata stalks with B. pumilus can convert substances therein into some ingredients with growth-promoting functions, and subsequently, these ingredients are extracted with ethanol to form a growth-promoting solution. The use of the growth-promoting solution in bar treatment and mycelia colonized material preparation can promote the growth of G. elata and the accumulation of functional ingredients. Moreover, planting substrates prepared with dregs after ethanol extraction can further provide nutrients for the growth of A. mellea. During the growth of the G. elata, mycelia of A. mellea provide the G. elata with nutrients needed for growth; use of the mycelia colonized material according to this technical solution can promote the supply of nutrients to the G. elata in the symbiotic process of the G. elata and the A. mellea, and facilitate the accumulation of secondary metabolites of the G. elata. G. elata cultivation is carried out by using the mycelia colonized material prepared by the technical solution, and the total content of GAS (C₁₃H₁₈O₇) and HBA (C₇H₈O₂) is ideal in the tubers of finished G. elata products obtained. In addition, B. pumilus, Bacillus subtilis, Bacillus circulans, and Bacillus mycoides are common microorganisms in the wild habitat of the G. elata. The inventors have tried to use these microorganisms to improve the planting quality of the G. elata. After screening, it is found that microbial biotransformation products obtained by treating the G. elata stalk with the B. pumilus have an optimal growth-promoting effect.

Planting G. elata has become the main source of Gastrodiae Rhizoma in China. While a large number of G. elata tubers are harvested, a large amount of by-products of the aerial parts (including stalks) of G. elata are produced every year, which are used for papermaking, biogas or feeds. The added value of the G. elata stalk is not fully developed. This technical solution adopts a B. pumilus fermentation method to transform the G. elata stalk into substances having a growth-promoting effect, which broadens the use of the G. elata stalk and creates conditions for the comprehensive utilization of G. elata resources.

To sum up, the mycelia colonized material prepared by this technical solution can play a positive role in promoting the growth of the G. elata and the accumulation of functional ingredients, and this technical solution further recycles waste parts in G. elata planting, turns waste into wealth, and further improves the comprehensive utilization rate of G. elata resources and the quality of cultivated G. elata.

Further, the fermentation culture may be conducted at 40-45° C. and 100 rpm for 48 h; during the fermentation culture, the materials to be treated may be sprayed with the B. pumilus suspension every 2-3 h; the materials to be treated and the B. pumilus suspension may have a consumption ratio of 100 g:15-20 mL in each spraying.

By adopting the above technical solution, under the above fermentation culture conditions, the B. pumilus may microbially transform ingredients in the G. elata stalk to produce substances with a growth-promoting effect. During the fermentation culture, spraying the B. pumilus suspension on the materials to be treated replenishes not only fresh spawns, but also water lost in the fermentation culture.

Further, the B. pumilus suspension may have an OD₆₀₀ value of 1.

By adopting the above technical solution, the G. elata stalk may be fully fermented using the B. pumilus suspension of the above concentration. If the OD₆₀₀ value is 1, the bacteria have ideal activity. In general, the OD₆₀₀ value of 1 represents a bacterial count of 2×10⁹ cfu/mL.

Further, the hot-dip extraction may be conducted at 80-90° C. for 10-12 h.

By adopting the above technical solution, ingredients having the growth-promoting effect in the fermented materials may be fully extracted.

Further, in the hot-dip extraction, the ethanol may have a concentration of 80% by volume, and the fermented materials and the ethanol may have a consumption ratio of 1 g:25 mL.

By adopting the above technical solution, the above concentration and consumption of the ethanol can ensure the extraction of the functional ingredients in the fermented materials by a solvent.

Further, the extract may be concentrated into an extractum having a relative density of 1.10, and every 1 g of the extractum may be reconstituted with 25 mL of water and subjected to the moist heat sterilization to obtain the growth-promoting solution.

By adopting the above technical solution, through concentration and sterilization, the ethanol in the material may be fully volatilized, and weed molds therein may be removed, so as to prevent the weed molds from affecting the growth of the G. elata and the A. mellea.

Further, after the moist heat sterilization, the residues may be dried at 50° C. until a water content is less than 5%, pulverized, and sieved through a No. 1 sieve to obtain the inoculation substrate.

By adopting the above technical solution, the residues are sterilized to remove the weed molds therein, so as to prevent the weed molds from affecting the growth of the G. elata and the A. mellea. The residues are dried and pulverized for subsequent mixing with A. mellea spawns.

Further, the A. mellea spawns and the inoculation substrate may have a mass ratio of (2-4):1.

By adopting the above technical solution and in accordance with the above ratio, the inoculation substrate may fully exert the growth-promoting effect thereof.

Further, a method for successively soaking the bars with the fish scale openings in the boiling water and the growth-promoting solution is as follows: soaking the bars in the boiling water for 5 min, cooling the bars, and soaking the bars in the growth-promoting solution for 12 h.

By adopting the above technical solution, microorganisms and eggs may be preliminarily removed from the bars by first soaking in the boiling water. Using the above soaking time may ensure that the bars can fully absorb growth-promoting substances in the growth-promoting solution.

Further, the bars each may be 8-10 cm in diameter and 60-70 cm in length; the fish scale openings may be arranged along the length of the bars, and adjacent fish scale openings may be 2-3 cm apart.

By adopting the above technical solution, the diameter of 8-10 cm is a conventional size of the mycelia colonized material (bar) in the prior art, and the length of 60-70 cm is suitable for the size of an incubator. The adjacent fish scale openings are 2-3 cm apart, in order to ensure that sufficient A. mellea is inoculated on the mycelia colonized material.

### DETAILED DESCRIPTION OF THE EMBODIMENTS

The present disclosure will be described in further detail below with reference to examples, but implementations of the present disclosure are not limited thereto. Unless otherwise specified, the technical means used in the following examples and experimental examples are conventional means well known to those skilled in the art, and the materials and reagents used can be commercially available.

The present disclosure will be described in further detail below with reference to the specific implementations.

### EXAMPLE 1

(1) Mycelia colonized material preparation: Branches of Quercus acutissima Carruth. were selected to make mycelia colonized materials. Specifically, fresh branches of Q. acutissima with a diameter of 8-10 cm were selected, and the branches were cut into bars with a length of about 60-70 cm. Some fish scale openings were cut out on the bars. The fish scale openings were arranged along the length of the bar, and adjacent fish scale openings were 2-3 cm apart. Subsequently, the bars were soaked in boiling water for 5 min, taken out and cooled; the bars were soaked in a growth-promoting solution for 12 h to obtain the bars after growth promotion treatment. The bars after growth promotion treatment were air-dried until the water content of the bars was reduced to 70%. Subsequently, a knife was used to pry open the fish scale openings, a little finger-sized A. mellea spawn was put in each fish scale opening, and the inoculation substrate was put at the same time, where the mass ratio of the inoculation substrate to the A. mellea spawn was 1:2 (for easy operation, generally, the inoculation substrate is applied on the outside of the A. mellea spawn, and then put in the fish scale opening). After the inoculation was completed, the fish scale openings were pressed hard, and the mycelia colonized material preparation was completed. After mycelia colonized materials were obtained, it was necessary to carry out the steps of cultivating G. elata seed bulbs or finished G. elata products as soon as possible.

G. elata stalks discarded in G. elata planting were used for the preparation of the growth-promoting solution and the inoculation substrate, and specific methods were as follows:

Fresh G. elata stalks were cut up to obtain materials to be treated. The materials to be treated were spread on a culture tray with a thickness of 3-4 cm, and a B. pumilus suspension was sprayed on the surface of the materials to be treated. The consumption ratio of the materials to be treated to the B. pumilus suspension was 100 g:20 mL. Subsequently, the tray was placed on a 40° C. incubation shaker, cultured at 100 rpm for 48 h (a gauze was needed to cover the materials to be treated), and sprayed with an equal volume of the B. pumilus suspension every 3 h. After fermentation, fermented materials were obtained. The method for obtaining the B. pumilus suspension was as follows: a 5% inoculum of B. pumilus Meyer and Gottheil (ATCC 7061) was inoculated on a conventional LB agar plate, and cultured at a constant temperature of 37° C. and 100 rpm until the OD₆₀₀ value was about 1. The culture was stopped, and the B. pumilus suspension was obtained.

The fermented materials were placed in 80% (v/v) ethanol, and 25 mL of ethanol was added per 1 g of the fermented materials to soak at 90° C. for 10 h; the fermented materials were filtered to achieve solid-liquid separation, and residues and an extract were obtained. The extract was concentrated into an extractum with a relative density of about 1.10 by conventional vacuum distillation, and the extractum was fully dissolved with pure water (the consumption ratio of the extractum to the pure water was 1 g:25 mL) and placed at 121° C. and 0.1 MPa for moist heat sterilization for 30 min to obtain the growth-promoting solution. The residues were placed at 121° C. and 0.1 MPa for moist heat sterilization for 30 min, dried in a 50° C. oven to a constant weight, pulverized and sieved through a No. 1 sieve to obtain the inoculation substrate.

(2) Cultivation of G. elata seed bulbs: G. elata was planted in late October. First of all, a layer of 4-6 cm thick soil (the first soil layer) was spread in a planting box, and the mycelia colonized materials were spread on the bottom of the planting box (to form the first mycelia colonized material layer). The spacing between adjacent mycelia colonized materials was 1-2 cm. The soaked G. elata seeds were sown evenly, as well as Mycena purpureofusca spawns (G. elata germination fungus). The sowing rate of G. elata seeds was around 0.5 kg/m² (the weight was calculated based on the unsoaked seeds). The M. purpureofusca spawns were cut into small pieces and sown, with a sowing rate of around 0.3 kg/m². The soil with a thickness of 1-2 cm (the second soil layer) was covered, and the mycelia colonized materials were spread on the second soil layer (to form the second mycelia colonized material layer). The spacing between adjacent mycelia colonized materials was 3-4 cm. Finally, the second mycelia colonized material layer was covered with 5-7 cm thick soil (the third soil layer). The planting box was timely sprayed with a volume of water, so that the water content was controlled at about 60% in the planting box, and the temperature of the planting box was controlled at around 25° C. After 6-month cultivation, G. elata seed bulbs (namely bulbs, also known as baima) were obtained. Herein, the method for soaking G. elata seeds was to immerse the G. elata seeds with clean water, and the soaking time was 8 h.

(3) Cultivation of finished G. elata products: G. elata was planted in late April of the following year. First of all, a layer of 4-6 cm thick soil (the first soil layer) was spread in a planting box, and the mycelia colonized materials were spread on the bottom of the planting box (to form the first mycelia colonized material layer). The spacing between adjacent mycelia colonized materials was 1-2 cm. G. elata seed bulbs were evenly placed on the first mycelia colonized material layer (the spacing between adjacent G. elata seed bulbs was 7 cm) and covered with 5-7 cm thick soil (the second soil layer). The planting box was timely sprayed with a volume of water, so that the water content was controlled at about 60% in the planting box, and the temperature of the planting box was controlled at around 25° C. After 7-month cultivation, finished G. elata products were obtained, and their tubers were collected for experimental studies.

(4) Finished product detection: Functional ingredients of the G. elata tubers obtained in this example were detected, and a total of 10 samples (10 G. elata tubers) were selected for detection. The detection method was carried out under Assay of Gastrodiae Rhizoma in the Pharmacopoeia of the People's Republic of China.

The conditions of high performance solution chromatography (HPLC) were as follows: octadecylsilane-bonded silica gel was used as the packing agent; acetonitrile-0.05% phosphoric acid solution (3:97) was used as the mobile phase; the detection wavelength was 220 nm.

The preparation method of the reference solution was as follows: a quantity of GAS reference substance and a quantity of HBA reference substance were accurately weighed, and acetonitrile-water (3:97) mixture was added to prepare a mixture containing 50 μg of GAS and 25 μg of HBA per 1 mL.

The preparation method of the test solution was as follows: About 2 g of G. elata tuber powder (sieved through a No. 3 sieve) was accurately weighed in a stoppered conical flask, accurately supplemented with 50 mL of dilute ethanol, weighed, sonicated (power 120 W, frequency 40 kHz) for 30 min, let cool, and weighed again, the lost weight was made up with dilute ethanol; after filtration, 10 mL of subsequent filtrate was accurately measured and concentrated to near dryness without alcohol smell; the residue was dissolved with an acetonitrile-water (3:97) mixture, transferred to a 25 mL measuring flask, diluted to volume with the acetonitrile-water (3:97) mixture, shaken well, and filtered to obtain a subsequent filtrate. The G. elata tuber powder was obtained by first drying the G. elata tuber at a constant temperature of 50° C. to a constant weight and then pulverizing.

The assay was as follows: accurately, 5 μL each of the reference solution and the test solution was pipetted and injected into a liquid chromatograph for assay, respectively. The assay results are shown in Table 1.

### EXAMPLE 2

This example was basically the same as Example 1, except that the preparation methods of the growth-promoting solution and the inoculation substrate, which were specifically as follows:

Fresh G. elata stalks were cut up to obtain materials to be treated. The materials to be treated were spread on a culture tray with a thickness of 3-4 cm, and a B. pumilus suspension was sprayed on the surface of the materials to be treated. The consumption ratio of the materials to be treated to the B. pumilus suspension was 100 g:15 mL. Subsequently, the tray was placed on a 45° C. incubation shaker, cultured at 100 rpm for 48 h, and sprayed with an equal volume of the B. pumilus suspension every 2 h. After fermentation, fermented materials were obtained. The method for obtaining the B. pumilus suspension was as follows: a 5% inoculum of B. pumilus Meyer and Gottheil (ATCC 7061) was inoculated on a conventional LB agar plate, and cultured until the OD₆₀₀ value was 1. The culture was stopped, and the B. pumilus suspension was obtained.

The fermented materials were placed in 80% (v/v) ethanol, and 25 mL of ethanol was added per 1 g of the fermented materials to soak at 80° C. for 12 h; the fermented materials were filtered to achieve solid-liquid separation, and residues and an extract were obtained. The extract was concentrated into an extractum with a relative density of about 1.10, and the extractum was fully dissolved with pure water (the consumption ratio of the extractum to the pure water was 1 g:25 mL) and placed at 121° C. and 0.1 MPa for moist heat sterilization for 30 min to obtain the growth-promoting solution. The residues were placed at 121° C. and 0.1 MPa for moist heat sterilization for 30 min, dried in a 50° C. oven to a constant weight, pulverized and sieved through a No. 1 sieve to obtain the inoculation substrate.

### EXAMPLE 3

This example was basically the same as Example 1, except that the mass ratio of the inoculation substrate to the A. mellea spawn was 1:4.

### Comparative Example 1

This comparative example was basically the same as Example 1, except that the bars were not soaked in the growth-promoting solution, and the preparation process of the mycelia colonized material was specifically as follows:

Branches of Q. acutissima were selected to make mycelia colonized materials. Specifically, fresh branches of Q. acutissima with a diameter of 8-10 cm were selected, and the branches were cut into bars with a length of about 70 cm. Some fish scale openings were cut out on the bars. Subsequently, the bars were soaked in boiling water for 5 min, taken out and cooled. The bars were air-dried until the water content of the bars was reduced to 70%. Subsequently, a knife was used to pry open the fish scale openings, a little finger-sized A. mellea spawn was put in each fish scale opening. After the inoculation was completed, the fish scale openings were pressed hard, and the mycelia colonized material preparation was completed. After mycelia colonized materials were obtained, it was necessary to carry out the steps of cultivating G. elata seed bulbs or finished G. elata products as soon as possible.

### Comparative Example 2

This comparative example was basically the same as Example 1, except that B. pumilus was equivalently replaced with B. subtilis (Ehrenberg) Cohn (ATCC 6051).

### Comparative Example 3

This comparative example was basically the same as Example 1, except that B. pumilus was equivalently replaced with B. circulans Jordan (ATCC 4513).

### Comparative Example 4

This comparative example was basically the same as that of Example 1, except that B. pumilus was equivalently replaced with B. mycoides Flugge (ATCC 19647).

### Comparative Example 5

This comparative example was basically the same as Example 1, except the following technical procedure:

The fermented materials were placed in pure water, and 25 mL of pure water was added per 1 g of the fermented materials to soak at 90° C. for 10 h; the fermented materials were filtered to achieve solid-liquid separation, and residues and an extract were obtained. The extract was concentrated into an extractum with a relative density of about 1.10, and the extractum was fully dissolved with pure water (the consumption ratio of the extractum to the pure water was 1 g:25 mL) and placed at 121° C. and 0.1 MPa for moist heat sterilization for 30 min to obtain the growth-promoting solution. The residues were placed at 121° C. and 0.1 MPa for moist heat sterilization for 30 min, dried in a 50° C. oven until the water content of <5% (w/w), pulverized and sieved through a No. 1 sieve to obtain the inoculation substrate.

The G. elata tubers obtained by Examples 1 to 3 and Comparative Examples 1 to 5 were detected (in order to ensure the parallelism of the experiment, all G. elata seeds used in Examples 1 to 3 and Comparative Examples 1 to 5 were from the same batch). The results are shown in Table 1, the total amount of GAS (C₁₃H₁₈O₇) and HBA (C₇H₈O₂) is calculated based on the mass fractions of both substances in the dry product. The “total content” in the table refers to the total amount of GAS and HBA.

Table 1: The assay results of the G. elata tubers of Examples 1 to 3 and Comparative Examples 1 to 5 (the mean total content (%) is expressed as the mean±standard deviation (SD))

| | | | | Comparative | Comparative | Comparative | Comparative | Comparative |
|---|---|---|---|---|---|---|---|---|
| Sample No. | Example 1 | Example 2 | Example 3 | Example 1 | Example 2 | Example 3 | Example 4 | Example 5 |
| Total | 1.036 | 1.113 | 1.022 | 0.621 | 0.879 | 0.579 | 1.032 | 0.566 |
| content of | | | | | | | | |
| sample 1 | | | | | | | | |
| (%) | | | | | | | | |
| Total | 1.001 | 1.259 | 1.057 | 0.557 | 0.964 | 0.641 | 0.977 | 0.857 |
| content of | | | | | | | | |
| sample 2 | | | | | | | | |
| (%) | | | | | | | | |
| Total | 1.157 | 0.967 | 0.936 | 0.349 | 0.647 | 0.743 | 0.876 | 0.472 |
| content of | | | | | | | | |
| sample 3 | | | | | | | | |
| (%) | | | | | | | | |
| Total | 1.081 | 0.826 | 1.297 | 0.282 | 0.698 | 0.581 | 0.943 | 0.954 |
| content of | | | | | | | | |
| sample 4 | | | | | | | | |
| (%) | | | | | | | | |
| Total | 1.173 | 0.975 | 0.899 | 0.493 | 0.872 | 0.943 | 0.844 | 0.813 |
| content of | | | | | | | | |
| sample 5 | | | | | | | | |
| (%) | | | | | | | | |
| Total | 0.981 | 1.027 | 0.976 | 0.497 | 0.913 | 0.871 | 0.629 | 0.623 |
| content of | | | | | | | | |
| sample 6 | | | | | | | | |
| (%) | | | | | | | | |
| Total | 0.859 | 1.033 | 1.076 | 0.857 | 0.762 | 0.698 | 0.574 | 0.767 |
| content of | | | | | | | | |
| sample 7 | | | | | | | | |
| (%) | | | | | | | | |
| Total | 0.976 | 1.051 | 0.973 | 0.367 | 0.497 | 0.595 | 0.693 | 0.921 |
| content of | | | | | | | | |
| sample 8 | | | | | | | | |
| (%) | | | | | | | | |
| Total | 1.137 | 0.873 | 0.827 | 0.437 | 0.556 | 0.794 | 0.743 | 0.637 |
| content of | | | | | | | | |
| sample 9 | | | | | | | | |
| (%) | | | | | | | | |
| Total | 0.824 | 0.927 | 0.966 | 0.369 | 0.614 | 0.945 | 0.882 | 0.679 |
| content of | | | | | | | | |
| sample 10 | | | | | | | | |
| (%) | | | | | | | | |
| Mean total | 1.023 ± | 1.005 ± | 1.001 ± | 0.483 ± | 0.740 ± | 0.739 ± | 0.819 ± | 0.729 ± |
| content | 0.119 | 0.124 | 0.127 | 0.167 | 0.162 | 0.144 | 0.153 | 0.159 |
| (%) | | | | | | | | |

As can be seen from the experimental results in Table 1, the total amount of GAS (C₁₃H₁₈O₇) and HBA (C₇H₈O₂) is ideal in the tubers of the finished G. elata products in Examples 1 to 3 obtained by adopting the organic planting method of G. elata according to this technical solution. This shows that fermentation of G. elata stalks with B. pumilus can convert substances therein into some ingredients with growth-promoting functions, and subsequently, these ingredients are extracted with ethanol to form a growth-promoting solution. The use of the growth-promoting solution in bar treatment and mycelia colonized material preparation can promote the growth of G. elata and the accumulation of functional ingredients. Moreover, planting substrates prepared with dregs after ethanol extraction can further provide nutrients for the growth of A. mellea. During the growth of the G. elata, mycelia of A. mellea provide the G. elata with nutrients needed for growth; use of the mycelia colonized material according to this technical solution can promote the supply of nutrients to the G. elata in the symbiotic process of the G. elata and the A. mellea, and facilitate the accumulation of secondary metabolites of the G. elata. Because the growth-promoting solution and the inoculation substrate according to this solution were not used in Comparative Example 1, the total content of GAS and HBA was lower in the obtained G. elata. In Comparative Examples 2 to 4, B. subtilis, B. circulans, and B. mycoides were used to ferment the G. elata stalks, but the effect was not as good as that of B. pumilus. B. pumilus, B. subtilis, B. circulans, and B. mycoides are common microorganisms in the wild habitat of the G. elata. The inventors have tried to use these microorganisms to improve the planting quality of the G. elata. However, the experiment found that there were differences in fermentation effect among these Bacillus strains, and B. pumilus had a significantly better effect than others (t-test, P<0.01). In Comparative Example 5, pure water was used to extract the fermented materials, and the effect was not very satisfactory, which indicated that most of the ingredients with growth-promoting effect were alcohol-soluble and ethanol extraction was more suitable.

The above are only examples of the present disclosure, and common knowledge such as well-known specific technical solutions and/or characteristics in the solutions are not described too much herein. It should be noted that those skilled in the art may further make several variations and improvements without departing from the technical solution of the present disclosure, which should also be deemed as falling within the protection scope of the present disclosure and will not affect the implementation effect of the present disclosure and the practicability of the patent. The protection scope claimed in this application shall be based on contents of claims, and disclosure in the specification such as the detailed description may be used to interpret the contents of the claims.

What is claimed is:

1. An organic planting method of Gastrodia elata, comprising the process of mycelia colonized material preparation: cutting a branch of Quercus acutissima into bars; cutting out fish scale openings on the bars to obtain bars with the fish scale openings; successively soaking the bars with the fish scale openings in boiling water and a growth-promoting solution to obtain bars after growth promotion treatment; putting Amillariella mellea spawns and an inoculation substrate into each fish scale opening of the bars after the growth promotion treatment to obtain a mycelia colonized material;
wherein the growth-promoting solution and the inoculation substrate are prepared by the following methods: cutting up a fresh Gastrodia elata stalk to obtain materials to be treated, spraying a Bacillus pumilus suspension on the materials to be treated, and conducting fermentation culture to obtain fermented materials; using ethanol as a solvent to conduct hot-dip extraction on the fermented material, and conducting solid-liquid separation to obtain residues and an extract; conducting moist heat sterilization, drying and pulverization on the residues to obtain the inoculation substrate; and conducting concentration, reconstitution and moist heat sterilization on the extract to obtain the growth-promoting solution.

2. The organic planting method of Gastrodia elata according to claim 1, wherein the fermentation culture is conducted at 40-45° C. and 100 rpm for 48 h; during the fermentation culture, the materials to be treated are sprayed with the Bacillus pumilus suspension every 2-3 h; the materials to be treated and the Bacillus pumilus suspension have a consumption ratio of 100 g:15-20 mL in each spraying.

3. The organic planting method of Gastrodia elata according to claim 2, wherein the Bacillus pumilus suspension has an OD₆₀₀ value of 1.

4. The organic planting method of Gastrodia elata according to claim 3, wherein the hot-dip extraction is conducted at 80-90° C. for 10-12 h.

5. The organic planting method of Gastrodia elata according to claim 4, wherein in the hot-dip extraction, the ethanol has a concentration of 80% by volume, and the fermented materials and the ethanol have a consumption ratio of 1 g:25 mL.

6. The organic planting method of Gastrodia elata according to claim 5, wherein the extract is concentrated into an extractum having a relative density of 1.10, and every 1 g of the extractum is reconstituted with 25 mL of water and subjected to the moist heat sterilization to obtain the growth-promoting solution.

7. The organic planting method of Gastrodia elata according to claim 6, wherein after the moist heat sterilization, the residues are dried at 50° C. until a water content is less than 5%, pulverized, and sieved through a No. 1 sieve to obtain the inoculation substrate.

8. The organic planting method of Gastrodia elata according to claim 7, wherein the Amillariella mellea spawns and the inoculation substrate have a mass ratio of (2-4):1.

9. The organic planting method of Gastrodia elata according to claim 8, wherein a method for successively soaking the bars with the fish scale openings in the boiling water and the growth-promoting solution is as follows: soaking the bars in the boiling water for 5 min, cooling the bars, and soaking the bars in the growth-promoting solution for 12 h.

10. The organic planting method of Gastrodia elata according to claim 9, wherein the bars each are 8-10 cm in diameter and 60-70 cm in length; the fish scale openings are arranged along the length of the bars, and adjacent fish scale openings are 2-3 cm apart.
